(19) United States
(12) Patent Application Publication
(10) Pub. No.: US 2026/0137689 A1
(43) Pub. Date: May 21, 2026

(54) INTEGRATION OF MOLECULAR MECHANISMS IN THE STRIATUM AS A COMBINATION DRUG STRATEGY FOR THE TREATMENT OF PSYCHIATRIC AND NEUROLOGICAL DISORDERS IN WHICH ANHEDONIA OR MOTIVATION-RELATED DYSFUNCTION EXISTS

(71) Applicant: Alto Neuroscience, Inc., Mountain View, CA (US)

(72) Inventors: Amit Etkin, Mountain View, CA (US); Dan Segal, Mountain View (AU)

(21) Appl. No.: 19/450,399

(22) Filed: Jan. 15, 2026

Related U.S. Application Data
(63) Continuation of application No. 17/648,538, filed on Jan. 20, 2022.
(60) Provisional application No. 63/199,723, filed on Jan. 20, 2021.

(51) Int. Cl. A61K 31/522; A61K 31/16; A61K 31/428; A61K 31/44; A61K 31/4985; A61K 31/501; A61P 25/24

(52) U.S. Cl. CPC A61K 31/522; A61K 31/16; A61K 31/428; A61K 31/44; A61K 31/4985; A61K 31/501; A61P 25/24

(57) ABSTRACT

The present invention relates to the use of a combination of two or more of a D2 agonist, an adenosine A2A receptor antagonist, a histamine H3 antagonist or inverse agonist, a mGluR5 receptor antagonist, or a nicotinic α₄-β₂ and/or α₇ receptor agonist to increase D2 dopaminergic molecular signaling in the striatum for the treatment of psychiatric or neurological disorders in which anhedonia or motivation-related dysfunction exists (such as major depressive disorder, bipolar I or II disorder, post-traumatic stress disorder, addiction, anhedonia or motivation-related aspects of schizophrenia (e.g. negative symptoms) and Parkinson's disease (e.g. non-motor features such as depression and apathy)).

### CROSS-REFERENCE TO RELATED APPLICATIONS

This application is a continuation of U.S. patent application Ser. No. 17/648,538, filed Jan. 20, 2022, which claims the benefit of U.S. Provisional Application No. 63/199,723 filed Jan. 20, 2021. The aforementioned applications are hereby incorporated by reference in their entirety.

### FIELD OF THE INVENTION

The present invention relates to the use of a combination of two or more of a D2 agonist, an adenosine A2A receptor antagonist, a histamine H3 antagonist or inverse agonist, a mGluR5 receptor antagonist, or a nicotinic α4-β2 and/or α7 receptor agonist to increase D2-related dopaminergic molecular signaling in the striatum for the treatment of psychiatric and neurological disorders in which anhedonia or motivation-related dysfunction exists (such as major depressive disorder, bipolar I disorder, post-traumatic stress disorder, addiction, anhedonia or motivation-related aspects of schizophrenia (e.g. negative symptoms), as well as Parkinson's disease).

### BACKGROUND

There are many psychiatric and neurological disorder in which depressive symptoms, lack of experience of pleasure (anhedonia) or motivation-related dysfunction exist. These are often difficult-to-treat features of these diseases and are predictive of a chronic and disabling course of illness. A need exists for more effective treatment in depression and diseases or disorders with a depressive, anhedonia, motivational and/or cognitive impairment component, given that even with the most comprehensive treatment regimen, only 43% of depressed patients achieve sustained remission over a one-year period (1) and this is the disorder in which the largest number of treatment options exist. Similar clinical needs exist in other related conditions in which depressive symptoms, lack of experience of pleasure (anhedonia) or anhedonia, motivation-related impairments exists. These conditions include major depressive disorder, bipolar depression (such as bipolar I disorder), post-traumatic stress disorder, addiction, anhedonia or motivation-related aspects of schizophrenia (e.g., negative symptoms) and Parkinson's Disease. Importantly, these different depression-related symptoms or areas of dysfunction co-occur and may be functionally related. For example, a patient with major depression may report depressed mood and lack of motivation. Likewise, the same symptoms may be reported by patients diagnosed with other conditions in which similar impairments may co-occur, such as bipolar depression, post-traumatic stress disorder or addiction. Though schizophrenia is often thought of with respect to prominent hallucinations and delusions, the depression-like negative symptoms are often the greater source of long-term disability and functional impairment. Similarly, though Parkinson's Disease involves prominent motor dysfunction, it also frequently has highly disabling non-motor features such as depression and apathy. Hence, a treatment approach that encompasses these multiple and related functional systems would be both of importance to any one of these clinical conditions, and equally may be applicable across them.

Preclinical and clinical studies have implicated the striatum and dopaminergic function as important for depression or aspects of its symptoms (e.g. anhedonia). For example, PET studies of various aspects of dopaminergic signaling have identified abnormalities in depressed patients (2-4), and functional imaging studies have described abnormal brain activation in striatal subregions with a prominent role for dopamine in emotion and emotionally-relevant behavior (5-8). Extensive characterization of neuroanatomy has furthermore delineated regions of the striatum (composed of the caudate nucleus and putamen), which take part in different functional circuits, as part of cortico-striato-thalamic loops (9-11). Of particular note, dorsal striatal regions have been linked to motor functions, while ventral striatum (also called the nucleus accumbens) has been linked to emotional functions such as reward, pleasure or reinforcement.

While it is presently believed that dopaminergic signaling plays an important role in depression, how this view can produce effective dopamine-based interventions for depression is uncertain due to seemingly inconclusive and contradictory findings:
The U.S. Food and Drug Administration (FDA) has approved multiple dopamine D2 receptor antagonists for the treatment of depression, which block dopamine action at the D2 receptor (particularly in the striatum). These drugs were initially developed for the treatment of schizophrenia.
Other work in smaller-scale studies used the D2/D3 agonist pramipexole for treating depression. Though results were variable across studies, meta-analytic summaries have indicated effectiveness for depression (12, 13).
Further confounding an understanding of how dopamine signaling can be therapeutically leveraged in depression, the canonical dopamine precursor treatment, L-DOPA (which gets converted in the brain to dopamine), is not an effective antidepressant (14).
Cocaine, which leads to a strong surge in dopamine, is likewise a drug of abuse and not an antidepressant. Therefore, there is no clear strategy for development of striatal dopamine-targeting treatments for depression.

Further complicating matters is that striatal function has typically been described in terms of a “direct” pathway and an “indirect” pathway (9-11, 15). This concept was derived from work on motor control by dorsal striatal regions and links D1 receptor function to the direct pathway, and D2 receptor function to the indirect pathway. The direct pathway has been shown to activate motor behavior while the indirect pathway inhibits motor behavior. When dopamine binds the D1 receptor, it activates the direct pathway, while when it binds the D2 receptor it inhibits the indirect pathway. The net effect of dopamine action is therefore to increase motor output. As such, in Parkinson's disease, where dopaminergic signaling is impoverished, motor output is reduced unless the patient is treated with a pro-dopaminergic drug.

The extension of this work to the limbic ventral striatum, however, is not straightforward. It has been argued that the limbic analogy to control of motor function in the striatum is such that D1-containing neurons encode positive valence, reward or pleasure, while D2-containing neurons encode negative valence or aversion (16-19). However, more recent work has shown that both D1- and D2-containing neurons can encode positive and negative stimuli through patterns of activity (20-25). As such, while it is still presently believed that dopaminergic signaling plays an important role in depression, how this view can produce effective dopamine-based interventions for depression is uncertain. Equally uncertain is the degree to which, if at all (given the L-DOPA results above), results from dopaminergic interventions in the motor domain can be generalized to the emotional domain relevant for depression.

One potential insight comes from the perspective that antipsychotic medications (D2 antagonists) and D2/D3 agonists like pramipexole have both shown evidence of efficacy. Thus, it may be that an improved ability to control D2-related neural signaling in the striatum is important.

Notably, it is not only a question of efficacy, but also whether an effective dose is possible to deliver, how long it takes to achieve that dose and with what level of side effects. For example, while one study showed superiority of pramipexole over placebo for depression, it targeted a total daily dose of 3 mg (if tolerated) but achieved only an average of 1.35 mg per day and a very modest clinical effect size at the cost of greater side effects with pramipexole than placebo (26). It is, in fact, well-appreciated that pro-dopaminergic drugs carry a very significant risk of side effects which limit tolerability, requiring that dosing be very gradually escalated over many weeks (27, 28). Indeed, in another positive study for pramipexole, the results at higher doses were not even statistically analyzable due to the high dropout rate (28), and an increased likelihood of dropout was confirmed in a meta-analysis when compared to conventional antidepressants (12). Strikingly, in a study in which pramipexole was better than the conventional antidepressant sertraline for the treatment of depression in Parkinson's disease, dose titration took seven weeks for pramipexole (29). This is much slower than titration for conventional antidepressants (such as selective serotonin reuptake inhibitors), which can achieve a therapeutic dose in 1-2 weeks while minimizing side effects. Thus, there is a need for D2-based therapeutics that achieve greater overall efficacy at lower doses or with fewer side effects, and which can be titrated more quickly to an effective dose range.

### SUMMARY OF THE INVENTION

One embodiment of the present invention is a pharmaceutical composition (e.g., an oral composition such as an oral tablet or oral solution) comprising at least two of a D2 agonist (such as a D2/D3 agonist), an adenosine A2A receptor antagonist, a histamine H3 antagonist or inverse agonist, a mGluR5 receptor antagonist, or a nicotinic α4-β2 and/or α7 receptor agonist. For instance, the pharmaceutical composition may comprise (a) an adenosine A2A receptor antagonist and (b) at least one of a histamine H3 antagonist or inverse agonist, a mGluR5 receptor antagonist, a nicotinic α4-β2 and/or α7 receptor agonist, and a D2 agonist.

In one embodiment, the composition comprises (a) an adenosine A2A receptor antagonist and (b) a D2 agonist (such as a D2/D3 agonist). In one preferred embodiment, the composition comprises (a) istradefylline or a pharmaceutically acceptable salt thereof (such as istradefylline free base) and (b) pramipexole or a pharmaceutically acceptable salt thereof (such as pramipexole free base). For instance, the composition may comprise from about 5 to about 40 mg of istradefylline or the equivalent amount of a pharmaceutically acceptable salt of istradefylline (e.g., from about 5 to about 40 mg of istradefylline free base) and from about 0.25 to about 3 mg of pramipexole or the equivalent amount of a pharmaceutically acceptable salt of pramipexole (e.g., from about 0.25 to about 3 mg of pramipexole free base).

In another embodiment, the composition comprises (a) an adenosine A2A receptor antagonist and (b) a mGluR5 receptor antagonist. In one preferred embodiment, the composition comprises (a) istradefylline or a pharmaceutically acceptable salt thereof (such as istradefylline free base) and (b) acamprosate or a pharmaceutically acceptable salt thereof (such as acamprosate calcium). For instance, the composition may comprise from about 300 to about 1800 mg of acamprosate or the equivalent amount of a pharmaceutically acceptable salt of acamprosate (for instance, 333 to about 1998 mg of acamprosate calcium) and from about 5 to about 40 mg of istradefylline or the equivalent amount of a pharmaceutically acceptable salt of istradefylline (e.g., from about 5 to about 40 mg of istradefylline free base).

In yet another embodiment, the composition comprises (a) an adenosine A2A receptor antagonist, (b) a mGluR5 receptor antagonist, and (c) a D2 agonist (such as a D2/D3 agonist).

In yet another embodiment, the composition comprises (a) an adenosine A2A receptor antagonist and (b) a nicotinic α₄-β₂ and/or α₇ receptor agonist (such as a nicotinic α₄-β₂ and α₇ receptor agonist). In one preferred embodiment, the composition comprises (a) istradefylline or a pharmaceutically acceptable salt thereof (such as istradefylline free base) and (b) varenicline or a pharmaceutically acceptable salt thereof (such as varenicline tartrate). For instance, the composition may comprise from about 5 to about 40 mg of istradefylline or the equivalent amount of a pharmaceutically acceptable salt of istradefylline (e.g., from about 5 to about 40 mg of istradefylline free base) and from about 0.25 to about 3 mg of varenicline or the equivalent amount of a pharmaceutically acceptable salt of varenicline (such as varenicline tartrate).

In yet another embodiment, the composition comprises (a) an adenosine A2A receptor antagonist and (b) a histamine H3 antagonist or inverse agonist. In one preferred embodiment, the composition comprises (a) istradefylline or a pharmaceutically acceptable salt thereof (such as istradefylline free base) and (b) irdabisant or a pharmaceutically acceptable salt thereof (such as irdabisant hydrochloride). For instance, the composition may comprise from about 5 to about 40 mg of istradefylline or the equivalent amount of a pharmaceutically acceptable salt of istradefylline (e.g., from about 5 to about 40 mg of istradefylline free base) and an amount of irdabisant or a pharmaceutically acceptable salt thereof (e.g., irdabisant hydrochloride) equivalent to about 1 μg to about 500 μg of irdabisant hydrochloride. In another preferred embodiment, the composition comprises (a) istradefylline or a pharmaceutically acceptable salt thereof (such as istradefylline free base) and (b) pitolisant or a pharmaceutically acceptable salt thereof. For instance, the composition may comprise from about 5 to about 40 mg of istradefylline or the equivalent amount of a pharmaceutically acceptable salt of istradefylline (e.g., from about 5 to about 40 mg of istradefylline free base) and an amount of pitolisant or a pharmaceutically acceptable salt thereof (e.g., pitolisant hydrochloride) equivalent to about 2 to about 40 mg of pitolisant hydrochloride.

In one preferred embodiment, the adenosine A2A receptor antagonist in the pharmaceutical compositions described herein is selected from istradefylline and pharmaceutically acceptable salts thereof (such as istradefylline free base).

In yet another embodiment, the composition comprises (a) a mGluR5 receptor antagonist and (b) a D2 agonist (such as a D2/D3 agonist). In one preferred embodiment, the composition comprises (a) acamprosate or a pharmaceutically acceptable salt thereof (such as acamprosate calcium) and (b) pramipexole or a pharmaceutically acceptable salt thereof (such as pramipexole free base). For instance, the composition may comprise from about 300 to about 1800 mg of acamprosate or the equivalent amount of a pharmaceutically acceptable salt of acamprosate (for instance, 333 to about 1998 mg of acamprosate calcium) and from about 0.25 to about 3 mg of pramipexole or the equivalent amount of a pharmaceutically acceptable salt of pramipexole (such as from about 0.25 to about 3 mg of pramipexole free base).

In yet another embodiment, the composition comprises (a) a D2 agonist and (b) a histamine H3 antagonist or inverse agonist. In one preferred embodiment, the composition comprises (a) pramipexole or a pharmaceutically acceptable salt thereof (e.g., pramipexole free base) and (b) irdabisant or a pharmaceutically acceptable salt thereof (such as irdabisant hydrochloride). For instance, the composition may comprise from about 0.25 to about 3 mg of pramipexole or the equivalent amount of a pharmaceutically acceptable salt of pramipexole (e.g., pramipexole free base) and an amount of irdabisant or a pharmaceutically acceptable salt thereof (e.g., irdabisant hydrochloride) equivalent to about 1 μg to about 500 μg of irdabisant hydrochloride. In another preferred embodiment, the composition comprises (a) pramipexole or a pharmaceutically acceptable salt thereof (e.g., pramipexole free base) and (b) pitolisant or a pharmaceutically acceptable salt thereof. For instance, the composition may comprise from about 0.25 to about 3 mg of pramipexole or the equivalent amount of a pharmaceutically acceptable salt of pramipexole (e.g., pramipexole free base) and an amount of pitolisant or a pharmaceutically acceptable salt thereof (e.g., pitolisant hydrochloride) equivalent to about 2 to about 40 mg of pitolisant hydrochloride.

In any of the embodiments described herein, the pharmaceutical composition may include an effective amount of the recited components (such as components (a) and (b) or components (a) through (c)) to treat the intended disorder, such as (a) depression (such as major depressive disorder or bipolar I disorder), (b) a psychiatric or neurological disorder in which anhedonia or motivation-related dysfunction exists, or (c) one or more symptoms associated with depression, anhedonia, or motivation-related impairments. In another embodiment, pharmaceutical composition may include an effective amount of the recited components (such as components (a) and (b) or components (a) through (c)) to increase D2 dopaminergic molecular signaling.

Another embodiment is a method of treating (a) depression (such as major depressive disorder or bipolar I disorder), (b) a psychiatric or neurological disorder in which anhedonia or motivation-related dysfunction exists, or (c) one or more symptoms associated with depression, anhedonia, or motivation-related impairments in a subject in need thereof comprising administering to the subject an effective amount of a pharmaceutical composition of the present invention. In one embodiment, an effective amount of the pharmaceutical composition is administered to increase D2 dopaminergic molecular signaling.

Yet another embodiment is a method of treating (a) depression (such as major depressive disorder or bipolar I disorder), (b) a psychiatric or neurological disorder in which anhedonia or motivation-related dysfunction exists, or (c) one or more symptoms associated with depression, anhedonia, or motivation-related impairments in a subject in need thereof comprising administering to the subject an effective amount of at least two of a D2 agonist, an antagonist of the adenosine A2A receptor, a histamine H3 antagonist or inverse agonist, an antagonist of the metabotropic glutamate mGluR5 receptor or an agonist of the nicotinic α4-β2 and/or α7 receptor to increase D2 dopaminergic molecular signaling.

In one embodiment, the method comprises administering an effective amount of (a) an adenosine A2A receptor antagonist and (b) at least one of a histamine H3 antagonist or inverse agonist, a mGluR5 receptor antagonist, a nicotinic α4-β2 and/or α7 receptor agonist, and a D2 agonist.

In one embodiment, the method comprises administering an effective amount of (a) an adenosine A2A receptor antagonist and (b) a D2 agonist (such as a D2/D3 agonist). In one preferred embodiment, the method comprises administering an effective amount of (a) istradefylline or a pharmaceutically acceptable salt thereof (such as istradefylline free base) and (b) pramipexole or a pharmaceutically acceptable salt thereof (such as pramipexole free base). For instance, in one embodiment, the method comprises administering from about 5 to about 40 mg per day of istradefylline or the equivalent amount of a pharmaceutically acceptable salt of istradefylline (e.g., from about 5 to about 40 mg per day of istradefylline free base) and from about 0.25 to about 3 mg per day of pramipexole or the equivalent amount of a pharmaceutically acceptable salt of pramipexole (e.g., from about 0.25 to about 3 mg per day of pramipexole free base).

In another embodiment, the method comprises administering an effective amount of (a) an adenosine A2A receptor antagonist and (b) mGluR5 receptor antagonist. In one preferred embodiment, the method comprises administering an effective amount of (a) istradefylline or a pharmaceutically acceptable salt thereof (such as istradefylline free base) and (b) acamprosate or a pharmaceutically acceptable salt thereof (such as acamprosate calcium). For instance, in one embodiment, the method comprises administering from about 300 to about 1800 mg per day of acamprosate or the equivalent amount of a pharmaceutically acceptable salt of acamprosate (for instance, 333 to about 1998 mg per day of acamprosate calcium) and from about 5 to about 40 mg per day of istradefylline or the equivalent amount of a pharmaceutically acceptable salt of istradefylline (e.g., from about 5 to about 40 mg per day of istradefylline free base).

In yet another embodiment, the method comprises administering an effective amount of (a) an adenosine A2A receptor antagonist, (b) a mGluR5 receptor antagonist, and (c) a D2 agonist.

In yet another embodiment, the method comprises administering an effective amount of (a) an adenosine A2A receptor antagonist and (b) a nicotinic α₄-β₂ and/or a receptor agonist (such as a nicotinic α₄-β₂ and α₇ receptor agonist or a nicotinic α₇ receptor agonist). In one preferred embodiment, the method comprises administering an effective amount of (a) istradefylline or a pharmaceutically acceptable salt thereof (such as istradefylline free base) and (b) varenicline or a pharmaceutically acceptable salt thereof (such as varenicline tartrate). For instance, in one embodiment, the method comprises administering from about 5 to about 40 mg per day of istradefylline or the equivalent amount of a pharmaceutically acceptable salt of istradefylline (e.g., from about 5 to about 40 mg per day of istradefylline free base) and from about 0.25 to about 3 mg per day of varenicline or the equivalent amount of a pharmaceutically acceptable salt of varenicline (such as varenicline tartrate).

In yet another embodiment, the method comprises administering an effective amount of (a) an adenosine A2A receptor antagonist and (b) a histamine H3 antagonist or inverse agonist. In one preferred embodiment, the method comprises administering an effective amount of (a) istradefylline or a pharmaceutically acceptable salt thereof (such as istradefylline free base) and (b) irdabisant or a pharmaceutically acceptable salt thereof (such as irdabisant hydrochloride). For instance, in one embodiment, the method comprises administering from about 5 to about 40 mg per day of istradefylline or the equivalent amount of a pharmaceutically acceptable salt of istradefylline (e.g., from about 5 to about 40 mg per day of istradefylline free base) and an amount of irdabisant or a pharmaceutically acceptable salt thereof (e.g., irdabisant hydrochloride) equivalent to about 1 μg to about 500 μg of irdabisant hydrochloride per day. In another preferred embodiment, the method comprises administering an effective amount of (a) istradefylline or a pharmaceutically acceptable salt thereof (such as istradefylline free base) and (b) pitolisant or a pharmaceutically acceptable salt thereof (such as pitolisant hydrochloride). For instance, in one embodiment, the method comprises administering from about 5 to about 40 mg per day of istradefylline or the equivalent amount of a pharmaceutically acceptable salt of istradefylline (e.g., from about 5 to about 40 mg per day of istradefylline free base) and an amount of pitolisant or a pharmaceutically acceptable salt thereof (e.g., pitolisant hydrochloride) equivalent to about 2 mg to about 40 mg of pitolisant hydrochloride per day.

In one preferred embodiment, the adenosine A2A receptor antagonist in the methods described herein is selected from istradefylline and pharmaceutically acceptable salts thereof (such as istradefylline free base).

In yet another embodiment, the method comprises administering an effective amount of (a) a mGluR5 receptor antagonist and (b) a D2 agonist (such as a D2/D3 agonist). In one preferred embodiment, the method comprises administering an effective amount of (a) acamprosate or a pharmaceutically acceptable salt thereof (such as acamprosate calcium) and (b) pramipexole or a pharmaceutically acceptable salt thereof (such as pramipexole free base). For instance, in one embodiment, the method comprises administering from about 300 to about 1800 mg per day of acamprosate or the equivalent amount of a pharmaceutically acceptable salt of acamprosate (for instance, 333 to about 1998 mg per day of acamprosate calcium) and from about 0.25 to about 3 mg per day of pramipexole or the equivalent amount of a pharmaceutically acceptable salt of pramipexole (such as from about 0.25 to about 3 mg per day of pramipexole free base).

In yet another embodiment, the method comprises administering an effective amount of (a) a histamine H3 antagonist or inverse agonist and (b) a D2 agonist (such as a D2/D3 agonist). In one preferred embodiment, the method comprises administering an effective amount of (a) irdabisant or a pharmaceutically acceptable salt thereof (such as irdabisant hydrochloride) and (b) pramipexole or a pharmaceutically acceptable salt thereof (such as pramipexole free base). For instance, in one embodiment, the method comprises administering an amount of irdabisant or a pharmaceutically acceptable salt thereof equivalent to about 1 μg to about 500 μg of irdabisant hydrochloride per day (for instance, 1 μg to about 500 μg per day of irdabisant hydrochloride) and from about 0.25 to about 3 mg per day of pramipexole or the equivalent amount of a pharmaceutically acceptable salt of pramipexole (such as from about 0.25 to about 3 mg per day of pramipexole free base). In another preferred embodiment, the method comprises administering an effective amount of (a) pitolisant or a pharmaceutically acceptable salt thereof (such as pitolisant hydrochloride) and (b) pramipexole or a pharmaceutically acceptable salt thereof (such as pramipexole free base). For instance, in one embodiment, the method comprises administering an amount of pitolisant or a pharmaceutically acceptable salt thereof equivalent to about 2 to about 40 mg of pitolisant hydrochloride per day (for instance, 2 to about 40 mg per day of pitolisant hydrochloride) and from about 0.25 to about 3 mg per day of pramipexole or the equivalent amount of a pharmaceutically acceptable salt of pramipexole (such as from about 0.25 to about 3 mg per day of pramipexole free base).

In another embodiment, the methods described herein may include administering an effective amount of the recited components (such as components (a) and (b) or components (a) through (c)) to increase D2 dopaminergic molecular signaling.

A preferred adenosine A2A receptor antagonist in any of the compositions or methods described herein is istradefylline, caffeine, theophylline, BIIB014, preladenant, ST-1535, ciforadenant, MSX-3, ZM 241385, SYN115, Lu AA47070, or a pharmaceutically acceptable salt thereof. A more preferred D2 agonist for the compositions and methods described herein is istradefylline or a pharmaceutically acceptable salt thereof (such as istradefylline free base).

The D2 agonist in any of the compositions or methods described herein may be a D2/D3 agonist, such as quinpirole, pramipexole, ropinirole, piribedil, rotigotine, pergolide, bromocriptine, apomorphine, cabergoline, ciladopa, dihydrexidine, dinapsoline, doxanthrine, epicriptine, lisuride, prophylnorapomorphine, quinagolide, roxindole, sumanirole or a pharmaceutically acceptable salt thereof. A more preferred D2 agonist for the compositions and methods described herein is pramipexole or a pharmaceutically acceptable salt thereof (such as pramipexole free base).

A preferred nicotinic α4-β2 and/or α7 receptor agonist for the compositions and methods described herein is varenicline, nicotine, 3-bromocytisine, cytisine, galantamine, epibatidine, epiboxidine, A-84543, A-366833, ABT-418, altinicline, dianicline, ispronicline, pozanicline, rivanicline, tebanicline, TC-1827, sazetidine A, tilorone, A-582941, AR-R17779, TC-1698, bradanicline, encenicline, GTS-21, PHA-543613, PNU-292987, PHA-709829, SSR-180711, tropisetron, WAY-317538, anabasine, PNU-120596, NS-1738, AVL-3288, A867744, ivermectine, BNC210, or a pharmaceutically acceptable salt thereof (such as varenicline tartrate). A more preferred nicotinic α4-β2 and/or α7 receptor agonist for the compositions and methods described herein is varenicline or a pharmaceutically acceptable salt thereof (such as varenicline tartrate).

A preferred mGluR5 antagonist for the compositions and methods described herein is acamprosate, basimglurant, mavoglurant, STX107, AZD2066, dipraglurant, or raseglurant or a pharmaceutically acceptable salt thereof. A more preferred mGluR5 antagonist for the compositions and methods described herein is acamprosate or a pharmaceutically acceptable salt thereof (such as acamprosate calcium).

A preferred H3 antagonist or inverse agonist for the compositions and methods described herein is irdabisant (CEP-26401), pitolisant, ABT-28, BF2.649, GSK-189254, GSK-239512, MK-0249, PF-3654746, or a pharmaceutically acceptable salt thereof (such as pitolisant hydrochloride or irdabisant hydrochloride).

### BRIEF DESCRIPTION OF THE DRAWINGS

FIG. 1 is a diagram of molecular mechanisms involved in the striatal indirect pathway that are relevant to the regulation of D2 neuron activity.

FIG. 2 is a bar graph of the time during a forced swim test after treatment with saline, 20 mg/kg for imipramine, 0.1 mg/kg for istradefylline, 0.17 mg/kg for varenicline, and a combination of 0.1 mg/kg for istradefylline and 0.17 mg/kg for varenicline.

### DETAILED DESCRIPTION OF THE INVENTION

Without being bound by any particular theory, the inventors theorize that a solution to this challenge is through harnessing additional molecular mechanisms relevant to dopamine and/or D2 signaling in the striatum. The D2 receptor, like other g-protein coupled receptors, are subject to desensitization. Thus, not only does harnessing additional molecular mechanisms improve overall efficacy, result through additive or synergistic action in lower doses of dopaminergic drugs that are particularly side-effect prone, and allow more rapid titration to an effective and tolerable dose, but may also present a more robust intervention from the neurobiological perspective. That is, by targeting two or more dopamine or D2-relevant signaling pathways it may be harder for the brain to counter the intended effects of the medication through processes such as desensitization—a long-standing and central challenge to central nervous systems therapeutics when they target a single molecular mechanism. Another advantage of a drug combination strategy that involves two or more distinct molecular targets (unlike the case of multiple drugs for the same target), is that their additive or synergistic action would be anatomically constrained to brain regions and neuron types that are impacted by both molecular processes. Put differently, while each individual drug in a combination has its own on-target effects and side effects, which are often dose-related, a drug combination would yield additive or synergistic effects in brain areas which are impacted by both drugs, which may serve to increase on-target effects and decrease side effects due solely to each individual drug. This is also because in a combination drug approach, lower doses may be possible for each individual component given their additive or synergistic on-target effects. This invention therefore details a novel combinatorial pharmacological strategy for robust and effective modulation of dopaminergic signaling for the purpose of the treatment of depression or diseases or disorders with a depressive component.

In order to identify potential drug combinations, we identified candidate molecular mechanisms relevant to dopamine or D2 signaling in the striatum. This was done based on information regarding where these molecules are expressed as well as any information suggestive of utility in depression. Additionally, we considered in our hypotheses known functional interactions between some of these molecules, which come exclusively from studies in either cellular or molecular preparations devoid of functional context (e.g., in a dissociated membrane preparation or in cultured neurons), or within the functional context of motor control. The utility of the drug combinations tested is unknown with respect to antidepressant activity, however, and for the reasons outlined above cannot be reasonably inferred from work on striatal motor circuitry. After having identified candidate molecular mechanisms, we then empirically tested whether their combinations resulted in antidepressant efficacy at doses where each individual drug was inactive. In doing so, we made surprising observations regarding effective drug combinations for the treatment of depression.

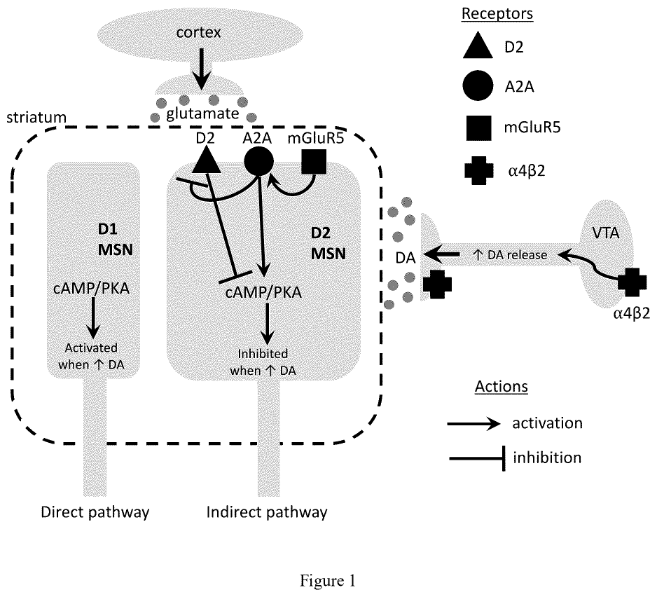

To frame our considerations, we summarize in FIG. 1 a reduced model of candidate molecular mechanisms relevant to the regulation of D2 neuron activity. FIG. 1 shows the inhibitory effect of A2A receptor stimulation on D2 receptor activity, as well as the activating effect of mGluR5 receptor engagement on A2A receptor activity. Furthermore, the A2A receptor activates signaling via cyclic AMP (cAMP) and protein kinase A (PKA) in D2-containing medium spiny neurons (MSN) in the indirect pathway, while the D2 receptor inhibits this signaling. The result of dopamine (DA) release from the ventral tegmental area (VTA) arriving at the striatum is that it activates D1 receptors on D1 MSNs, which increases cAMP/PKA signaling, and promotes influence of those neurons on their downstream targets. This is also sometimes termed the “Go pathway” as engagement of D1 MSNs promotes thalamic and cortical activation. By contrast, when DA arrives in the striatum it activates D2 receptors on D2 MSNs, which inhibit cAMP/PKA signaling in those neurons. This pathway is sometimes called the “NoGo pathway” and thus D2 receptor signaling would inhibit an inhibitory pathway on ultimate thalamic and cortical activity. The net result is therefore similar to D1 MSN activation (i.e. DA inhibiting the indirect pathway is aligned with the action of DA activating the direct pathway). DA release is furthermore under partial control from alpha4-beta2 (α₄β₂) nicotinic acetylcholine receptors in the VTA. As FIG. 1 shows, use of an A2A antagonist together with a D2 agonist will have a synergistic effect in inhibiting D2 MSN activity. Likewise use of an A2A antagonist with an mGluR5 antagonist, optionally in further combination with a D2 agonist, would have a synergistic effect in inhibiting D2 MSN activity. Replacing D2 agonist action with DA release by a nicotinic α₄β₂ and/or α₇ agonist, in combination with an A2A antagonist would then have a similar D2 MSN inhibitory effect.

Specifically, it has been reported that D2 receptors are in functional antagonism with the adenosine A2A receptors. D2 and A2A receptors structurally interact to form heteromers located on the dendritic spines of striatal medium spiny neurons (30, 31). The existence of this heteromer has been shown to result in internalization and desensitization of both receptors in the presence of D2 and A2A agonists (32). In other words, A2A activation is expected to lead to enhanced inactivation of D2 signaling.

The function of these interactions has often been studied in the context of animal models of Parkinson's disease, in which dopaminergic neurotransmission is reduced or removed. In these models, drugs or toxins are given to animals which either strongly block dopamine signaling or result in degeneration of dopaminergic neurons. One experiment using striatal slices from reserpinized rats found that in this context, an A2A agonist CGS 21680 decreased the affinity of the D2 receptor for the D2/D3 agonist quinpirole, while the A2A antagonist ZM 241385 increased the affinity of D2/D3 for quinpirole (33). Another study using similar methodologies found that a combination of the A2A antagonist MSX-3 and D2 agonist quinpirole led to both a greater magnitude and duration of GABAergic interneuron inhibition (34). Using a parkinsonian marmoset animal model, another study showed that the A2A antagonist istradefylline enhances the anti-parkinsonian activity of the dopamine agonists ropinirole and pergolide (35). In a similar manner, multiple A2A antagonists have been found to reverse a D2 antagonist or tetrabenazine (monoamine-depleter)-caused bias of animals to choose low-effort rewards instead of putting in more effort to receive greater rewards (36, 37). Though A2A antagonists, such as istradefylline, are not used for the treatment of depression, here we disclose the surprising combination of an A2A antagonist and a low dose of a D2/D3 agonist can be used to treat symptoms related to depression, anhedonia or motivation-related impairments. For example, use of 5-40 mg of istradefylline concurrently with 0.25-3 mg of pramipexole is one such combination. This combination can both lead to greater improvement in depressive symptoms and/or lead to lower side effects compared to use of a D2/D3 agonist such as pramipexole alone.

In addition to A2A, data also suggest that signaling at the metabotropic glutamate 5 (mGluR5) receptor is relevant to both A2A signaling and D2 signaling. Indeed, both A2A-mGluR5 and D2-A2A-mGluR5 heteromers have been identified (30). In one experiment, the mGluR5 agonist (RS)-2-chloro-5-hydroxyphenylglycine (CHPG) was able to counteract the effects of the D2/D3 agonist quinipirole on neuronal activity, evident based on release of their neurotransmitter gamma-aminobutyric acid (GABA), even though it was ineffective by itself on GABA release (38). These effects were also counteracted by the A2A antagonist ZM 241385. Here we disclose that combination of an mGluR5 antagonist with a low dose of a D2/D3 agonist can be used to treat symptoms related to depression, anhedonia or motivation-related impairments. Examples of mGluR5 antagonists include basimglurant, mavoglurant, dipraglurant, raseglurant, AZD2066 and STX107. Additionally, the drug acamprosate has been found to possess mGluR5 antagonist properties (39, 40). Thus, for example, 333 mg-1998 mg of acamprosate calcium with 0.25-3 mg of pramipexole is one such combination. This combination can both lead to greater improvement in depressive symptoms and/or lead to lower side effects compared to use of a D2/D3 agonist such as pramipexole alone.

Without being bound by a particular theory, given the fact that A2A and mGluR5 both functionally antagonize signaling at the D2 receptor, the inventors theorized that antagonists for both receptors, when given together at individually ineffective doses, could result in an antidepressant response by virtue of their combined action on endogenous dopamine. That is, that instead of giving a D2 agonist, because both paths for D2 inhibition (i.e., A2A and mGluR5) are antagonized at the same time by separate drugs, the intended effects on D2 inhibition can be achieved by the combination's effect on endogenous levels of dopamine. Encouraging us to test this hypothesis are motor system findings that show that a combination of A2A and mGluR5 antagonists reverse Parkinsonian deficits in mice (41, 42). Here we disclose that a combination of A2A and mGluR5 antagonists can be used to treat symptoms related to depression, anhedonia or motivation-related impairments. For example, 333 mg-1998 mg of acamprosate calcium with 5-40 mg of istradefylline is one such combination.

It has also been reported that the histamine H3 receptor forms functional heterodimers or functionally interact with the D2 receptor and A2A receptor (48-51). The H3 receptor, like the D2 receptor, negatively couples to CAMP signaling in D2 MSNs. Moreover, blocking H3 activity increases the affinity of the D2 receptor to its ligands, and potentiates the effect of D2 agonists. The H3 receptor heteromerizes with the A2A receptor, against which it has an opposing influence (i.e. H3 activation decreased the affinity of the A2A receptor for its ligand). Thus, the H3 receptor has functional properties similar to that of the A2A receptor with respect to their shared opposite effects on D2 signaling, but the H3 receptor additionally exerts inhibitory effects on the A2A receptor.

Without being bound by a particular theory, given the D2-inhibiting effects of the H3 receptor, the inventors theorized that H3 antagonists or inverse agonists in combination with a D2 agonist could increase D2 signaling and result in an antidepressant effect. This combination is conceptually similar to that of an A2A antagonist and D2 agonist, as disclosed above. Here we disclose that a combination of a D2/D3 agonist and an H3 antagonist or inverse agonist can be used to treat symptoms related to depression, anhedonia or motivation-related impairments. For example, 0.25-3 mg of pramipexole with either 4.45-35.6 mg of pitolisant or 1-500 μg of irdabisant are two such combinations.

Similarly, a combination of an H3 antagonist or inverse agonist with an A2A antagonist could act to increase D2 activity even though H3 blockade may indirectly lead to an increase in A2A signaling. Here we disclose that a combination of an A2A antagonist and an H3 antagonist or inverse agonist can be used to treat symptoms related to depression, anhedonia or motivation-related impairments. For example, 5-40 mg of istradefylline with either 4.45-35.6 mg of pitolisant or 1-500 μg of irdabisant are two such combinations.

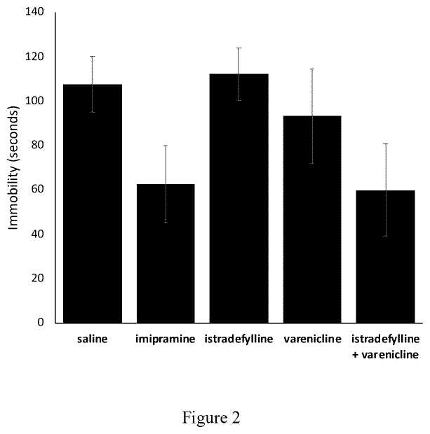

Without being bound by a particular theory, the inventors theorized that an increase in dopamine release, when coupled with A2A antagonism, could result in an antidepressant response-essentially aiming to replace direct D2 agonism with a drug that leads to its release and to an increase in D2/D3 receptor availability. The nicotinic α₄β₂ and/or α₇ agonist varenicline has been found to increase dopamine release in rodents and in humans (43, 44), as well as increase D2/D3 receptor availability in rodents (45, 46). Varenicline can also improve motor functioning in Parkinson's models (47). Of note, however, varenicline has been approved in humans for smoking cessation, and initially carried a black box for increased risk for development of depression, a seemingly opposite outcome to our goal. To determine whether such a combination is viable, we conducted forced swim tests in C57/BL6 mice. In this test, chemicals with antidepressant properties have been found to reduce the amount of time the animal spends immobile in a pool of water in which it can neither reach the bottom nor escape. As seen in FIG. 2, we found that combination of doses of istradefylline and varenicline that were by themselves ineffective nonetheless resulted in an effective antidepressant response in this test. The magnitude of this effect was furthermore comparable to the decrease in immobility with imipramine, a well-known antidepressant. Therefore, despite the black box warning having been included on varenicline when it was approved, the combination of an A2A antagonist and α₄β₂ and α₇ agonist (such as varenicline) is surprisingly effective at treating symptoms related to depression, anhedonia or motivation-related impairments. One such example is 5 mg-40 mg of istradefylline together with 0.25-3 mg of varenicline.

### Definitions

“D2 agonist” refers to an agonist of D2, such as quinpirole, pramipexole, ropinirole, piribedil, rotigotine, pergolide, bromocriptine, apomorphine, cabergoline, ciladopa, dihydrexidine, dinapsoline, doxanthrine, epicriptine, lisuride, prophylnorapomorphine, quinagolide, roxindole, sumanirole. The D2 agonist can be a D2/D3 agonist.

“D2/D3 agonist” refers to a selective agonist of both the D2 and D3 receptors. Suitable D2/D3 agonists include, but are not limited to, quinpirole, pramipexole, ropinirole, piribedil, rotigotine, pergolide, bromocriptine, apomorphine, cabergoline, ciladopa, dihydrexidine, dinapsoline, doxanthrine, epicriptine, lisuride, prophylnorapomorphine, quinagolide, roxindole, sumanirole, and pharmaceutically acceptable salts thereof.

“A2A antagonist” refers to an antagonist of the. Suitable A2A antagonists include, but are not limited to, istradefylline, caffeine, theophylline, BIIB014, preladenant, ST-1535, ciforadenant, MSX-3, ZM 241385, SYN115, Lu AA47070 and pharmaceutically acceptable salts thereof.

“Nicotinic α4-β2 and/or α7 receptor agonist” or “a nicotinic alpha4beta2 or alpha7 receptor agonist” refers to an agonist of the nicotinic α4-β2 and/or α7 nicotinic receptor containing these subunits. Suitable nicotinic α₄-β₂ receptor agonists include, but are not limited to, varenicline, nicotine, 3-bromocytisine, cytisine, galantamine, epibatidine, epiboxidine, A-84543, A-366833, ABT-418, altinicline, dianicline, ispronicline, pozanicline, rivanicline, tebanicline, TC-1827, sazetidine A or a pharmaceutically acceptable salt thereof (such as varenicline tartrate). Suitable α7 nicotinic receptor agonist include, but are not limited to varenicline, tilorone, A-582941, AR-R17779, TC-1698, bradanicline, encenicline, GTS-21, PHA-543613, PNU-292987, PHA-709829, SSR-180711, tropisetron, WAY-317538, anabasine, epiboxidine, PNU-120596, NS-1738, AVL-3288, A867744, ivermectine, BNC210 or a pharmaceutically acceptable salt thereof (such as varenicline tartrate). Unless otherwise specified, the recited amounts of “varenicline or a pharmaceutically acceptable salt thereof” refers to an equivalent amount of varenicline free base. 0.5 mg varenicline free base is equivalent to 0.85 mg of varenicline tartrate.

“mGluR5 antagonist” refers to a metabotropic glutamate receptor type 5 (mGluR5) antagonist. Suitable mGluR5 antagonists include, but are not limited to, acamprosate, basimglurant, mavoglurant, STX107, AZD2066, dipraglurant, or raseglurant, and pharmaceutically acceptable salts thereof (such as acamprosate calcium). Unless otherwise specified, the recited amounts of “acamprosate or a pharmaceutically acceptable salt thereof” refers to an equivalent amount of acamprosate free base. 300 mg acamprosate free base is equivalent to 333 mg of acamprosate calcium.

“H3 antagonist” or “H3 inverse agonist” refers to a compound that blocks activity at the H3 receptor. Suitable H3 antagonists or inverse agonists include, but are not limited to, pitolisant, ABT-28, BF2.649, CEP-26401 (irdabisant), GSK-189254, GSK-239512, MK-0249, PF-3654746 and pharmaceutically acceptable salts thereof (such as pitolisant hydrochloride or irdabisant hydrochloride).

Unless otherwise specified, the term “about” in the context of a numerical value or range refers to ±10% of the numerical value or range recited.

As used herein, “effective” as in an amount effective to achieve an end means the quantity of a component that is sufficient to yield an indicated therapeutic response without undue adverse side effects (such as toxicity, irritation, or allergic response) commensurate with a reasonable benefit/risk ratio when used in the manner of this disclosure. The specific effective amount varies with such factors as the particular condition being treated, the physical condition of the patient, the type of mammal being treated, the duration of the treatment, the nature of concurrent therapy (if any), and the specific formulations employed and the structure of the compounds or its derivatives.

As used herein, to “treat” or “treating” encompasses, e.g., inducing inhibition, regression, or stasis of a disorder and/or disease, e.g. depression, or alleviating, lessening, suppressing, inhibiting, reducing the severity of, eliminating or substantially eliminating, or ameliorating a symptom of the disease or disorder.

As used herein, the terms “subject” and “patient” are used interchangeably and refer to a human patient unless indicated otherwise.

Diagnosis of various mental and psychological disorders, including depression may be found, e.g., in the Diagnostic and Statistical Manual of Mental Disorders (5th Ed. DSM-5, American Psychiatric Association, 2013).

### Methods of Treatment

Each active ingredient (such as a D2 agonist, an adenosine A2A receptor antagonist, a mGluR5 receptor antagonist, or a nicotinic α4-β2 and/or α7 receptor agonist) may be administered by any route, such as orally, nasally, transdermally, rectally, percutaneously or by parenteral injection. A preferred route of administration is oral. The active ingredients may be administered in the form of a tablet, capsule, granules, or oral liquid.

The methods and pharmaceutical compositions described herein may be used to treat (a) depression (such as major depressive disorder or bipolar I disorder), (b) a psychiatric or neurological disorder in which anhedonia or motivation-related dysfunction exists, or (c) one or more symptoms associated with depression, anhedonia, or motivation-related impairments. The types of depression which may be treated include, but are not limited to, major depressive disorder, treatment resistant depression, residual depressive symptoms and dysthymia. Psychiatric or neurological disorders in which anhedonia or motivation-related dysfunction exists which may be treated include, but are not limited to, depression as part of bipolar I or bipolar II disorders, drug addiction, post-traumatic stress disorder, schizophrenia (in particular associated negative symptoms), or Parkinson's disease (non-motor features such as depression or apathy). Symptoms associated with depression which may be treated include, but are not limited to, depressed mood, blunted affect, anhedonia, alexithymia, and apathy. Anhedonia or motivation-related impairments which may be treated include, but are not limited to, inability to engage in previously rewarding experiences, reduced social interest or drive, inattentiveness to social inputs, reduced psychomotor activity, excessive sleep, avoidance of activities or social interactions, and decreased appetite.

Generally, the amount of the active ingredients to be administered is sufficient to increase D2 dopaminergic molecular signaling in the striatum. In one embodiment, the amount of each component to be administered daily can be as shown in the table below.

| Active Ingredient | Category | Range | Preferred Range |
|---|---|---|---|
| Istradefylline or a | A2A receptor | Amount equivalent to | Amount equivalent to |
| pharmaceutically | antagonist | about 5 to about 40 mg | about 5 to about 20 mg |
| acceptable salt | | istradefylline free | (such as about 10 |
| thereof | | base daily (preferably | to about 20 mg) |
| | | once daily) | istradefylline free |
| | | | base daily (preferably |
| | | | once daily) |
| Varenicline or a | nicotinic α₄-β₂ partial | Amount equivalent to | Amount equivalent to |
| pharmaceutically | agonist | about 0.25 to about 3 mg | about 0.5 to about 2 mg |
| acceptable salt | | varenicline free | or about 0.5 to |
| thereof (e.g., | | base daily (preferably | about 1 mg |
| varenicline tartrate) | | given once daily or in | varenicline free base |
| | | two divided doses) | (e.g., about 0.85 mg |
| | | | to about 3.42 mg or |
| | | | about 0.85 mg to |
| | | | about 1.71 mg of |
| | | | varenicline tartrate) |
| | | | daily (preferably |
| | | | given once daily or in |
| | | | two divided doses) |
| Acamprosate or a | mGluR5 antagonist | Amount equivalent to | Amount equivalent to |
| pharmaceutically | | about 300 to about | about 300 to about |
| acceptable salt | | 1800 mg daily of | 1200 mg of |
| thereof (e.g., | | acamprosate base or | acamprosate free |
| acamprosate calcium) | | the equivalent | base (e.g., 333 mg to |
| | | amount of its salt | 1,332 mg |
| | | (e.g., about 333 to | acamprosate calcium) |
| | | about 1,998 mg of | given once daily or in |
| | | acamprosate calcium) | two or three divided |
| | | given once daily or in | doses (for instance, |
| | | two or three divided | 600 mg acamprosate |
| | | doses (for instance, | base or the equivalent |
| | | 600 mg acamprosate | amount of its salt |
| | | base or the equivalent | given three times daily) |
| | | amount of its salt | |
| | | given three times daily) | |
| Pramipexole or a | D2/D3 agonist | Amount equivalent to | Amount equivalent to |
| pharmaceutically | | about 0.25 to about 3 mg | 0.5 to 2 mg or 0.5 to |
| acceptable salt | | pramipexole free | 1 mg pramipexole |
| thereof (e.g., | | base daily (given | free base daily (given |
| pramipexole | | once daily or in two | once daily or in two |
| dihydrochloride such | | or three divided doses) | or three divided doses) |
| as pramipexole | | | |
| dihydrochloride | | | |
| monohydrate) | | | |
| Pitolisant or a | H₃ antagonist or | An amount of | Amount equivalent to |
| pharmaceutically | inverse agonist | pitolisant or a | 4.45 mg to 17.8 mg |
| acceptable salt | | pharmaceutically | pitolisant free base |
| thereof (e.g., | | acceptable salt | (e.g., 5 to 20 mg of |
| pitolisant | | thereof equivalent to | pitolisant |
| hydrochloride) | | about 2 to about 40 | hydrochloride) daily |
| | | mg of pitolisant | (given once daily or |
| | | hydrochloride daily | in two divided doses) |
| | | (given once daily or | |
| | | in two divided doses) | |
| Irdabisant or a | H₃ antagonist or | An amount of | Amount equivalent to |
| pharmaceutically | inverse agonist | irdabisant or a | 5 μg to 250 μg |
| acceptable salt | | pharmaceutically | irdabisant HCl daily |
| thereof (e.g., | | acceptable salt | (given once daily or |
| irdabisant | | thereof equivalent to | in two divided doses) |
| hydrochloride) | | about 1 μg to about | |
| | | 500 μg of irdabisant | |
| | | hydrochloride daily | |
| | | (given once daily or | |
| | | in two divided doses) | |

In accordance with the practice of the invention, each active ingredient can be administered one or more times a day, daily, weekly, monthly or yearly.

### Pharmaceutical Compositions

The pharmaceutical composition can include one or more pharmaceutically acceptable excipients in addition to the active ingredients. The pharmaceutical composition may be suitable for any route of administration, such as nasal, rectal, intercisternal, buccal, intramuscular, intrasternal, intracutaneous, intrasynovial, intravenous, intraperitoneal, intraocular, periosteal, intra-articular injection, infusion, oral, topical, inhalation, parenteral, subcutaneous, implantable pump, continuous infusion, gene therapy, intranasal, intrathecal, intracerebroventricular, transdermal, or by spray, patch or injection.

The pharmaceutical composition may be formulated as a solid dosage form, such as capsules, pills, soft-gels, tablets, caplets, troches, wafer, sprinkle, or chewing for oral administration. The pharmaceutical composition may also be formulated as a liquid dosage form such as an elixir, suspension or syrup.

The pharmaceutical composition may also be presented in a dosage form for transdermal application (e.g., a patch or an ointment) or oral administration.

The pharmaceutical composition may be in a liquid dosage form or a suspension to be applied to nasal cavity or oral cavity using a dropper, a sprayer or a container. The pharmaceutical composition may be in a solid, salt or powder to be applied to nasal cavity or oral cavity using a sprayer, a forced air or a container.

The pharmaceutical acceptable excipient may be selected from pharmaceutically acceptable carriers, binders, diluents, adjuvants, or vehicles, such as preserving agents, fillers, polymers, disintegrating agents, glidants, wetting agents, emulsifying agents, suspending agents, sweetening agents, flavoring agents, perfuming agents, lubricating agents (such as magnesium stearate), acidifying agents, coloring agent, dyes, preservatives and dispensing agents. Such pharmaceutically acceptable excipients are described in the Handbook of Pharmaceutical Excipients, 6th Ed., Pharmaceutical Press and American Pharmaceutical Association (2009).

Pharmaceutically acceptable carriers are generally non-toxic to recipients at the dosages and concentrations employed and are compatible with other ingredients of the formulation. Examples of pharmaceutically acceptable carriers include water, saline, dextrose solution, ethanol, polyols, vegetable oils, fats, ethyl oleate, liposomes, waxes polymers, including gel forming and non-gel forming polymers, and suitable mixtures thereof. The carrier may contain minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulin; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

Examples of binders include, but are not limited to, microcrystalline cellulose and cellulose derivatives, gum tragacanth, glucose solution, acacia mucilage, gelatin solution, molasses, polyvinylpyrrolidone, povidone, crospovidone, sucrose and starch paste.

Examples of diluents include, but are not limited to, lactose, sucrose, starch, kaolin, salt, mannitol and dicalcium phosphate.

Examples of excipients include, but are not limited to, starch, surfactants, lipophilic vehicles, hydrophobic vehicles, pregelatinized starch, microcrystalline cellulose, lactose, milk sugar, sodium citrate, calcium carbonate, and dicalcium phosphate. Typical excipients for dosage forms such as a soft-gel include gelatin for the capsule and oils such as soy oil, rice bran oil, canola oil, olive oil, corn oil, and other similar oils; glycerol, polyethylene glycol liquids, and vitamin E TPGS as a surfactant.

Examples of disintegrating agents include, but are not limited to, complex silicates, croscarmellose sodium, sodium starch glycolate, alginic acid, corn starch, potato starch, bentonite, methylcellulose, agar and carboxymethylcellulose.

Examples of glidants include, but are not limited to, colloidal silicon dioxide, talc, corn starch.

Examples of wetting agents include, but are not limited to, propylene glycol monostearate, sorbitan monooleate, diethylene glycol monolaurate and polyoxyethylene laural ether.

Examples of lubricants include magnesium or calcium stearate, sodium lauryl sulphate, talc, starch, lycopodium and stearic acid as well as high molecular weight polyethylene glycols.

### EXAMPLES

### Example 1: Forced Swim Test with Istradefylline and Varenicline

To determine whether such a combination is viable, forced swim tests in C57/BL6 mice were conducted. The forced-swim test is used to assess depressive-like behavior in mice. The time spent immobile is considered a measure of depressive-like behavior. Immobility scores for each mouse were determined by manual scoring. In this test, chemicals with antidepressant properties have been found to reduce the amount of time the animal spends immobile in a pool of water in which it can neither reach the bottom nor escape.

As seen in FIG. 2, we found that combination of doses of istradefylline and varenicline that were by themselves ineffective nonetheless resulted in an effective antidepressant response in this test. The magnitude of this effect was furthermore comparable to the decrease in immobility with imipramine, a well-known antidepressant. Therefore, despite the black box warning having been included on varenicline when it was approved, the combination of an A2A antagonist and alpha4-beta2 agonist (such as varenicline) is a surprisingly effective at treating symptoms related to depression, anhedonia or motivation-related impairments.

### REFERENCES

1. Nelson J C. The STAR*D study: a four-course meal that leaves us wanting more. Am J Psychiatry. 2006; 163:1864-1866.
2. Pizzagalli D A, Berretta S, Wooten D, Goer F, Pilobello K T, Kumar P, Murray L, Beltzer M, Boyer-Boiteau A, Alpert N, El Fakhri G, Mechawar N, Vitaliano G, Turecki G, Normandin M. Assessment of Striatal Dopamine Transporter Binding in Individuals With Major Depressive Disorder: In Vivo Positron Emission Tomography and Postmortem Evidence. JAMA Psychiatry. 2019.
3. Schneier F R, Slifstein M, Whitton A E, Pizzagalli D A, Reinen J, McGrath P J, Iosifescu D V, Abi-Dargham A. Dopamine Release in Antidepressant-Naive Major Depressive Disorder: A Multimodal [(11) C]-(+)-PHNO Positron Emission Tomography and Functional Magnetic Resonance Imaging Study. Biol Psychiatry. 2018; 84:563-573.
4. Tiger M, Svensson J, Liberg B, Saijo T, Schain M, Halldin C, Farde L, Lundberg J. [(11) C] raclopride positron emission tomography study of dopamine-D2/3 receptor binding in patients with severe major depressive episodes before and after electroconvulsive therapy and compared to control subjects. Psychiatry Clin Neurosci. 2020; 74:263-269.
5. Barch D M, Pagliaccio D, Luking K. Mechanisms Underlying Motivational Deficits in Psychopathology: Similarities and Differences in Depression and Schizophrenia. Curr Top Behav Neurosci. 2016; 27:411-449.
6. Nestler E J, Carlezon W A, Jr. The mesolimbic dopamine reward circuit in depression. Biol Psychiatry. 2006; 59:1151-1159.
7. Whitton A E, Treadway M T, Pizzagalli D A. Reward processing dysfunction in major depression, bipolar disorder and schizophrenia. Curr Opin Psychiatry. 2015; 28:7-12.
8. Pizzagalli D A. Depression, stress, and anhedonia: toward a synthesis and integrated model. Annu Rev Clin Psychol. 2014; 10:393-423.
9. Haber S N, Behrens T E. The neural network underlying incentive-based learning: implications for interpreting circuit disruptions in psychiatric disorders. Neuron. 2014; 83:1019-1039.
10. Haber S N, Knutson B. The reward circuit: linking primate anatomy and human imaging. Neuropsychopharmacology. 2010; 35:4-26.
11. Haber S N. Corticostriatal circuitry. Dialogues Clin Neurosci. 2016; 18:7-21.
12. Tundo A, de Filippis R, De Crescenzo F. Pramipexole in the treatment of unipolar and bipolar depression. A systematic review and meta-analysis. Acta Psychiatr Scand. 2019; 140:116-125.
13. Szmulewicz A G, Angriman F, Samame C, Ferraris A, Vigo D, Strejilevich S A. Dopaminergic agents in the treatment of bipolar depression: a systematic review and meta-analysis. Acta Psychiatr Scand. 2017; 135:527-538.
14. Miguelez C, Berrocoso E, Mico J A, Ugedo L. L-DOPA modifies the antidepressant-like effects of reboxetine and fluoxetine in rats. Neuropharmacology. 2013; 67:349-358.
15. Alexander G E, Crutcher M D. Functional architecture of basal ganglia circuits: neural substrates of parallel processing. Trends Neurosci. 1990; 13:266-271.
16. Tai L H, Lee A M, Benavidez N, Bonci A, Wilbrecht L. Transient stimulation of distinct subpopulations of striatal neurons mimics changes in action value. Nat Neurosci. 2012; 15:1281-1289.
17. Hikida T, Kimura K, Wada N, Funabiki K, Nakanishi S. Distinct roles of synaptic transmission in direct and indirect striatal pathways to reward and aversive behavior. Neuron. 2010; 66:896-907.
18. Lobo M K, Covington H E, 3rd, Chaudhury D, Friedman A K, Sun H, Damez-Werno D, Dietz D M, Zaman S, Koo J W, Kennedy P J, Mouzon E, Mogri M, Neve R L, Deisseroth K, Han M H, Nestler E J. Cell type-specific loss of BDNF signaling mimics optogenetic control of cocaine reward. Science. 2010; 330:385-390.
19. Kravitz A V, Tye L D, Kreitzer A C. Distinct roles for direct and indirect pathway striatal neurons in reinforcement. Nat Neurosci. 2012; 15:816-818.
20. Cui G, Jun S B, Jin X, Pham M D, Vogel S S, Lovinger D M, Costa R M. Concurrent activation of striatal direct and indirect pathways during action initiation. Nature. 2013; 494:238-242.
21. Soares-Cunha C, Coimbra B, David-Pereira A, Borges S, Pinto L, Costa P, Sousa N, Rodrigues A J. Activation of D2 dopamine receptor-expressing neurons in the nucleus accumbens increases motivation. Nat Commun. 2016; 7:11829.
22. Natsubori A, Tsutsui-Kimura I, Nishida H, Bouchekioua Y, Sekiya H, Uchigashima M, Watanabe M, de Kerchove d′Exaerde A, Mimura M, Takata N, Tanaka K F. Ventrolateral Striatal Medium Spiny Neurons Positively Regulate Food-Incentive, Goal-Directed Behavior Independently of D1 and D2 Selectivity. J Neurosci. 2017; 37:2723-2733.
23. Soares-Cunha C, Coimbra B, Domingues A V, Vasconcelos N, Sousa N, Rodrigues A J. Nucleus Accumbens Microcircuit Underlying D2-MSN-Driven Increase in Motivation. eNeuro. 2018; 5.
24. Vicente A M, Galvao-Ferreira P, Tecuapetla F, Costa R M. Direct and indirect dorsolateral striatum pathways reinforce different action strategies. Curr Biol. 2016; 26: R267-269.
25. Soares-Cunha C, de Vasconcelos N A P, Coimbra B, Domingues A V, Silva J M, Loureiro-Campos E, Gaspar R, Sotiropoulos I, Sousa N, Rodrigues A J. Nucleus accumbens medium spiny neurons subtypes signal both reward and aversion. Mol Psychiatry. 2019.
26. Cusin C, Iovieno N, Iosifescu D V, Nierenberg A A, Fava M, Rush A J, Perlis R H. A randomized, double-blind, placebo-controlled trial of pramipexole augmentation in treatment-resistant major depressive disorder. J Clin Psychiatry. 2013; 74: e636-641.
27. Borovac J A. Side effects of a dopamine agonist therapy for Parkinson's disease: a mini-review of clinical pharmacology. Yale J Biol Med. 2016; 89:37-47.
28. Corrigan M H, Denahan A Q, Wright C E, Ragual R J, Evans D L. Comparison of pramipexole, fluoxetine, and placebo in patients with major depression. Depress Anxiety. 2000; 11:58-65.
29. Barone P, Scarzella L, Marconi R, Antonini A, Morgante L, Bracco F, Zappia M, Musch B, Depression/Parkinson Italian Study G. Pramipexole versus sertraline in the treatment of depression in Parkinson's disease: a national multicenter parallel-group randomized study. J Neurol. 2006; 253:601-607.
30. Cabello N, Gandia J, Bertarelli D C, Watanabe M, Lluis C, Franco R, Ferre S, Lujan R, Ciruela F. Metabotropic glutamate type 5, dopamine D2 and adenosine A2a receptors form higher-order oligomers in living cells. J Neurochem. 2009; 109:1497-1507.
31. Fuxe K, Agnati L F, Jacobsen K, Hillion J, Canals M, Torvinen M, Tinner-Staines B, Staines W, Rosin D, Terasmaa A, Popoli P, Leo G, Vergoni V, Lluis C, Ciruela F, Franco R, Ferre S. Receptor heteromerization in adenosine A2A receptor signaling: relevance for striatal function and Parkinson's disease. Neurology. 2003; 61: S19-23.
32. Hillion J, Canals M, Torvinen M, Casado V, Scott R, Terasmaa A, Hansson A, Watson S, Olah M E, Mallol J, Canela E I, Zoli M, Agnati L F, Ibanez C F, Lluis C, Franco R, Ferre S, Fuxe K. Coaggregation, cointernalization, and codesensitization of adenosine A2A receptors and dopamine D2 receptors. J Biol Chem. 2002; 277:18091-18097.
33. Floran B, Gonzalez B, Floran L, Erlij D, Aceves J. Interactions between adenosine A (2a) and dopamine D2 receptors in the control of [(3) H] GABA release in the globus pallidus of the rat. Eur J Pharmacol. 2005; 520:43-50.
34. Stromberg I, Popoli P, Muller C E, Ferre S, Fuxe K. Electrophysiological and behavioural evidence for an antagonistic modulatory role of adenosine A2A receptors in dopamine D2 receptor regulation in the rat dopamine-denervated striatum. Eur J Neurosci. 2000; 12:4033-4037.
35. Uchida S, Soshiroda K, Okita E, Kawai-Uchida M, Mori A, Jenner P, Kanda T. The adenosine A2A receptor antagonist, istradefylline enhances the anti-parkinsonian activity of low doses of dopamine agonists in MPTP-treated common marmosets. Eur J Pharmacol. 2015; 747:160-165.
36. Salamone J D, Correa M, Ferrigno S, Yang J H, Rotolo R A, Presby R E. The Psychopharmacology of Effort-Related Decision Making: Dopamine, Adenosine, and Insights into the Neurochemistry of Motivation. Pharmacol Rev. 2018; 70:747-762.
37. Lopez-Cruz L, Salamone J D, Correa M. Caffeine and Selective Adenosine Receptor Antagonists as New Therapeutic Tools for the Motivational Symptoms of Depression. Front Pharmacol. 2018; 9:526.
38. Beggiato S, Tomasini M C, Borelli A C, Borroto-Escuela D O, Fuxe K, Antonelli T, Tanganelli S, Ferraro L. Functional role of striatal A2A, D2, and mGlu5 receptor interactions in regulating striatopallidal GABA neuronal transmission. J Neurochem. 2016; 138:254-264.
39. Harris B R, Prendergast M A, Gibson D A, Rogers D T, Blanchard J A, Holley R C, Fu M C, Hart S R, Pedigo N W, Littleton J M. Acamprosate inhibits the binding and neurotoxic effects of trans-ACPD, suggesting a novel site of action at metabotropic glutamate receptors. Alcohol Clin Exp Res. 2002; 26:1779-1793.
40. Palucha-Poniewiera A, Pilc A. Involvement of mGlu5 and NMDA receptors in the antidepressant-like effect of acamprosate in the tail suspension test. Prog Neuropsychopharmacol Biol Psychiatry. 2012; 39:102-106.
41. Coccurello R, Breysse N, Amalric M. Simultaneous blockade of adenosine A2A and metabotropic glutamate mGlu5 receptors increase their efficacy in reversing Parkinsonian deficits in rats. Neuropsychopharmacology. 2004; 29:1451-1461.
42. Kachroo A, Orlando L R, Grandy D K, Chen J F, Young A B, Schwarzschild M A. Interactions between metabotropic glutamate 5 and adenosine A2A receptors in normal and parkinsonian mice. J Neurosci. 2005; 25:10414-10419.
43. Reperant C, Pons S, Dufour E, Rollema H, Gardier A M, Maskos U. Effect of the alpha4beta2* nicotinic acetylcholine receptor partial agonist varenicline on dopamine release in beta2 knock-out mice with selective re-expression of the beta2 subunit in the ventral tegmental area. Neuropharmacology. 2010; 58:346-350.
44. Di Ciano P, Guranda M, Lagzdins D, Tyndale R F, Gamaleddin I, Selby P, Boileau I, Le Foll B. Varenicline-Induced Elevation of Dopamine in Smokers: A Preliminary [(11) C]-(+)-PHNO PET Study. Neuropsychopharmacology. 2016; 41:1513-1520.
45. Crunelle C L, Schulz S, de Bruin K, Miller M L, van den Brink W, Booij J. Dose-dependent and sustained effects of varenicline on dopamine D2/3 receptor availability in rats. Eur Neuropsychopharmacol. 2011; 21:205-210.
46. Crunelle C L, de Wit TC, de Bruin K, Ramakers R M, van der Have F, Beekman F J, van den Brink W, Booij J. Varenicline increases in vivo striatal dopamine D2/3 receptor binding: an ultra-high-resolution pinhole [123I] IBZM SPECT study in rats. Nucl Med Biol. 2012; 39:640-644.
47. Tan R, Bolukbasi Hatip F, Acikalin O, Yamauchi A, Kataoka Y, Hatip-Al-Khatib I. Effect of varenicline on behavioral deficits in a rat model of Parkinson's disease induced by unilateral 6-hydroxydopamine lesion of substantia nigra. Behav Pharmacol. 2018; 29:327-335.
48. Ferrada C, et al., Neuropharmacology 55 (2): 190-7.
49. Ellenbroek B, et al., Trends Neurosci. 2014, 37 (4): 191-9.
50. Morales-Figueroa, G, et al., Purinergic Signal 2019, 15 (1): 85-93.
51. Marquez-Gomez, R, et al., Pharmacol Res. 2018, 129:515-525.

All references cited herein are hereby incorporated by reference.

1. A pharmaceutical composition comprising at least two of a D2 agonist, an adenosine A2A receptor antagonist, a histamine H3 antagonist or inverse agonist, a mGluR5 receptor antagonist, or a nicotinic α4-β2 and/or α7 receptor agonist.

2. The pharmaceutical composition of claim 1, wherein the composition comprises (a) an adenosine A2A receptor antagonist and (b) at least one of a histamine H3 antagonist or inverse agonist, a mGluR5 receptor antagonist, a nicotinic α4-β2 and/or α7 receptor agonist, and a D2 agonist.

3. The pharmaceutical composition of claim 1, wherein the composition comprises (a) an adenosine A2A receptor antagonist and (b) a D2 agonist.

4. The pharmaceutical composition of claim 3, wherein the composition comprises (a) istradefylline or a pharmaceutically acceptable salt thereof and (b) pramipexole or a pharmaceutically acceptable salt thereof.

5. The pharmaceutical composition of claim 4, wherein the composition comprises from about 5 to about 40 mg of istradefylline or the equivalent amount of a pharmaceutically acceptable salt of istradefylline and from about 0.25 to about 3 mg of pramipexole or the equivalent amount of a pharmaceutically acceptable salt of pramipexole.

6-8. (canceled)

9. The pharmaceutical composition of claim 1, wherein the composition comprises (a) an adenosine A2A receptor antagonist, (b) mGluR5 receptor antagonist, and (c) a D2 agonist.

10-20. (canceled)

21. The pharmaceutical composition of claim 1, wherein the composition comprises (a) a mGluR5 receptor antagonist and (b) a D2 agonist.

22. The pharmaceutical composition of claim 21, wherein the composition comprises (a) acamprosate or a pharmaceutically acceptable salt thereof and (b) pramipexole or a pharmaceutically acceptable salt thereof.

23. The pharmaceutical composition of claim 22, wherein the composition comprises from about 300 to about 1800 mg of acamprosate or the equivalent amount of a pharmaceutically acceptable salt of acamprosate and from about 0.25 to about 3 mg of pramipexole or the equivalent amount of a pharmaceutically acceptable salt of pramipexole.

24. The pharmaceutical composition of claim 1, wherein the composition comprises (a) a D2 agonist and (b) a histamine H3 antagonist or inverse agonist.

25. The pharmaceutical composition of claim 24, wherein the composition comprises (a) pramipexole or a pharmaceutically acceptable salt thereof and (b) irdabisant or a pharmaceutically acceptable salt thereof.

26. The pharmaceutical composition of claim 25, wherein the composition comprises (a) from about 0.25 to about 3 mg of pramipexole or the equivalent amount of a pharmaceutically acceptable salt of pramipexole and (b) an amount of irdabisant or a pharmaceutically acceptable salt thereof equivalent to about 1 μg to about 500 μg of irdabisant hydrochloride.

27. The pharmaceutical composition of claim 24, wherein the composition comprises (a) pramipexole or a pharmaceutically acceptable salt thereof and (b) pitolisant or a pharmaceutically acceptable salt thereof.

28. The pharmaceutical composition of claim 27, wherein the composition comprises (a) from about 0.25 to about 3 mg of pramipexole or the equivalent amount of a pharmaceutically acceptable salt of pramipexole and (b) an amount of pitolisant or a pharmaceutically acceptable salt thereof equivalent to about 2 mg to about 40 mg of pitolisant hydrochloride.

29. (canceled)

30. A method of treating (a) depression, (b) a psychiatric or neurological disorder in which anhedonia or motivation-related dysfunction exists, or (c) one or more symptoms associated with depression, anhedonia, or motivation-related impairments in a subject in need thereof comprising administering to the subject an effective amount of at least two of a D2 agonist, an antagonist of the adenosine A2A receptor, a histamine H3 antagonist or inverse agonist, an antagonist of the metabotropic glutamate mGluR5 receptor or an agonist of the nicotinic α4-β2 and/or α7 receptor.

31. The method of claim 30, wherein the method comprises administering an effective amount of (a) an adenosine A2A receptor antagonist and (b) at least one of a histamine H3 antagonist or inverse agonist, a mGluR5 receptor antagonist, a nicotinic α4-β2 receptor agonist, and a D2 agonist.

32. The method of claim 30, wherein the method comprises administering an effective amount of (a) an adenosine A2A receptor antagonist and (b) a D2 agonist.

33. The method of claim 32, wherein the method comprises administering an effective amount of (a) istradefylline or a pharmaceutically acceptable salt thereof and (b) pramipexole or a pharmaceutically acceptable salt thereof.

34. The method of claim 33, wherein the method comprises administering from about 5 to about 40 mg per day of istradefylline or the equivalent amount of a pharmaceutically acceptable salt of istradefylline and from about 0.25 to about 3 mg per day of pramipexole or the equivalent amount of a pharmaceutically acceptable salt of pramipexole.

35-37. (canceled)

38. The method of claim 30, wherein the method comprises administering an effective amount of (a) an adenosine A2A receptor antagonist, (b) mGluR5 receptor antagonist, and (c) a D2 agonist.

39-49. (canceled)

50. The method of claim 30, wherein the method comprises administering an effective amount of (a) a mGluR5 receptor antagonist and (b) a D2 agonist.

51. The method of claim 50, wherein the method comprises administering an effective amount of (a) acamprosate or a pharmaceutically acceptable salt thereof and (b) pramipexole or a pharmaceutically acceptable salt thereof.

52. The method of claim 51, wherein the method comprises administering from about 300 to about 1800 mg per day of acamprosate or the equivalent amount of a pharmaceutically acceptable salt of acamprosate and from about 0.25 to about 3 mg per day of pramipexole or the equivalent amount of a pharmaceutically acceptable salt of pramipexole.

53. The method of claim 30, wherein the method comprises administering an effective amount of (a) a D2 agonist and (b) a histamine H3 antagonist or inverse agonist.

54. The method of claim 53, wherein the method comprises administering an effective amount of (a) pramipexole or a pharmaceutically acceptable salt thereof and (b) irdabisant or a pharmaceutically acceptable salt thereof.

55. The method of claim 54, wherein the method comprises administering (a) from about 0.25 to about 3 mg per day of pramipexole or the equivalent amount of a pharmaceutically acceptable salt of pramipexole and (b) an amount of irdabisant or a pharmaceutically acceptable salt thereof equivalent to about 1 μg to about 500 μg of irdabisant hydrochloride per day.

56. The method of claim 53, wherein the method comprises administering an effective amount of (a) pramipexole or a pharmaceutically acceptable salt thereof and (b) pitolisant or a pharmaceutically acceptable salt thereof.

57. The method of claim 56, wherein the method comprises administering (a) from about 0.25 to about 3 mg per day of pramipexole or the equivalent amount of a pharmaceutically acceptable salt of pramipexole and (b) an amount of pitolisant or a pharmaceutically acceptable salt thereof equivalent to about 2 mg to about 40 mg of pitolisant hydrochloride per day.

58. (canceled)
